(19) United States
(12) Patent Application Publication
(10) Pub. No.: US 2005/0240181 A1
(43) Pub. Date: Oct. 27, 2005

(54) SPINAL IMPLANT CONNECTORS

(76) Inventors: Mark C. Boomer, Irvine, CA (US); Raymond F. Murphy, Attleboro, MA (US); Stephen Johnson, Greenville, RI (US); Bryan S. Jones, Norwood, MA (US); Thomas V. Doherty, Bellingham, MA (US); Wade Gotcher, Medfield, MA (US); Matthew Lake, Braintree, MA (US); Missoum Moumene, Newton, MA (US); J. Riley Hawkins, Cumberland, RI (US); William Kennefick, Plymouth, MA (US); Shawn Stad, Fall River, MA (US)

Correspondence Address: NUTTER MCCLENNEN & FISH LLP, WORLD TRADE CENTER WEST, 155 SEAPORT BOULEVARD, BOSTON, MA, 02210-2604, US

(21) Appl. No.: 10/709,246

(22) Filed: Apr. 23, 2004

(51) Int. Cl. A61B 17/58

(52) U.S. Cl. 606/61

(57) ABSTRACT

The present invention provides various methods and devices for mating spinal fixation elements, such as fixation rods, to various spinal anchoring devices, such as plates, hooks, bolts, wires, and screws. In an exemplary embodiment, an implantable spinal connector is provided having a clamp member with top and bottom portions that are connected to one another at a terminal end thereof, and that are adapted to seat a spinal fixation element there between. The top and bottom portions are preferably movable between an open position in which the top and bottom portions are spaced a distance apart from one another, and a closed position in which the portions are adapted to engage a spinal fixation element positioned there between. The connector can also include a bore extending through the top and bottom portions for receiving a locking mechanism that is effective to lock the top and bottom portions in the closed position to retain a spinal fixation element there between.

### FIELD OF THE INVENTION

The present invention relates to spinal instrumentation, and in particular to various connector devices for mating spinal fixation elements, such as spinal rods, to spinal anchoring devices, such as plates, hooks, bolts, wires, and screws, that are implanted in a patient's spinal system.

### BACKGROUND OF THE INVENTION

Treatment of some spinal injuries or disorders may involve the use of a spinal fixation element, such as a relatively rigid fixation rod, that is coupled to adjacent vertebrae by attaching the element to various anchoring devices, such as plates, hooks, bolts, wires, or screws. Often two rods are disposed on opposite sides of the spinous process in a substantially parallel relationship. The fixation rods can have a predetermined contour that has been designed according to the properties of the target implantation site, and once installed, the rods hold the vertebrae in a desired spatial relationship, until healing or spinal fusion has taken place, or for some longer period of time.

A variety of techniques have been developed for attaching a spinal rod to a vertebra, and each technique varies depending on the intended implanted location in the patient's spinal column. When surgery is performed in the cervical spine, for example, the proximal ends of the rods can be attached to the occiput by inserting a bone screw through a flattened, plate-like portion formed on the end of each rod. Other techniques utilize a spinal fixation plate having a yoke or receiver head that seats the spinal rod to attach the rod to the plate. A locking mechanism is utilizes to lock the rod to the plate, and the plate is attached to one or more vertebrae using one or more bone screws inserted therethrough.

While current techniques have been met with success, most do not allow the connection between the spinal rod and the anchoring device to be adjusted. For example, where the spinal rod is connected to a yoke on the fixation plate, the position of the rod is limited to being directly over the plate. Such a connection can also limit use of the device to specific regions of the spinal system, as the yoke does not provide a low-profile connection with the rod.

Accordingly, there remains a need for improved methods and devices for connecting spinal fixation elements, such as spinal rods, to spinal anchoring devices, such as plates, hooks, bolts, wires, and screws.

### BRIEF SUMMARY OF THE INVENTION

The present invention provides various embodiments of implantable spinal connectors for mating spinal fixation elements, such as spinal rods, to spinal anchoring devices, such as plates, hooks, bolts, wires, and screws. In one embodiment, an implantable spinal connector is provided having a clamp member with top and bottom portions that are connected to one another at a terminal end thereof. The top and bottom portions are movable between an open position in which the top and bottom portions are spaced a distance apart from one another, and a closed position in which the portions are adapted to engage a spinal fixation element disposed there between. The top and bottom portions also preferably include a bore extending therethrough for receiving a locking mechanism to lock the clamp member in the closed position.

The top and bottom portions of the connector member can be mated to one another using a variety of techniques. In one embodiment, the top and bottom portions can be hingedly connected to one another, and more particularly, a pin member can extend through bores formed in each of the top and bottom portions for allowing the portions to pivot thereabout. In another embodiment, the top and bottom portions can be integrally formed with one another, and they can be adapted to deform around a rod when the portions are in the closed position and the rod is locked therein. The top and bottom portions can also optionally be biased to either an open position or a closed position such that a force greater than the biasing force must be applied to move the top and bottom portions from the open position to the closed position or from the closed position to the open position.

In another embodiment of the present invention, a recess for seating a spinal fixation element is formed between the clamp members, preferably in at least one of the inferior surface of the top portion and the superior surface of the bottom portion, and the recess is adapted to seat a spinal fixation element therein when the top and bottom portions are in the closed position. The recess can have a variety of configurations, and it can be adapted to retain a variety of spinal fixation elements therein. In an exemplary embodiment, a recess is formed in each of the top and bottom portions, and each recess has a substantially concave shape such that the recesses together define a substantially cylindrical recess when the clamp member is in the closed position. In another embodiment, the recesses can have an elongate shape to allow the position of a rod disposed therein to be adjusted.

The connector mechanism can also include a locking mechanism that is adapted to lock the top and bottom portions in the closed position to retain a spinal fixation element there between. In one embodiment, at least a portion of the locking mechanism can extend through the bore formed in the top and bottom portions to lock the top and bottom portions in the closed position. The bore can have various shapes and sizes, but it preferably has either an elongate slotted configuration or a cylindrical shape. The locking mechanism can be, for example, a fastening element having a head and a threaded shaft. The threaded shaft can mate to corresponding threads formed within the bore in one or the top or bottom portions, and in an exemplary embodiment the threads on each of the shaft and in the bore in one of the top and bottom portions are left-handed threads. More preferably, the bore formed in the top portion is non-threaded for freely rotatably receiving the threaded shaft of the fastening element, and the bore formed in the bottom portion is threaded for mating with the threaded shaft of the fastening element. In an alternative embodiment, the threaded shaft can mate with a receiving element.

The present invention also provides an installation device for implanting a spinal connector. In one embodiment, the installation device includes first and second opposed arms that are pivotally coupled to one another and that are movable between an open position, wherein a distal portion of each arm is spaced a distance apart from one another, and a closed position, wherein the distal portion of each arm is adapted to seat and engage a spinal connector disposed there between. The distal portion of each arm can be substantially C-shaped to define a connector-receiving recess formed there between when the arms are in the closed position, and the distal portion of each arm can also include a connector-engaging member formed thereon and adapted to engage a connector positioned there between. The connector-engaging member can be, for example, a ridge that is adapted to extend between top and bottom portions of the spinal connector. The installation device can also include a locking mechanism coupled to each of the first and second arms and adapted to lock the arms in a fixed position relative to one another.

In another embodiment, an installation device for implanting a spinal connector is provided having an elongate shaft with a connector-receiving member formed on a distal end thereof and adapted to seat a spinal connector, and a driver shaft rotatably disposed through the elongate shaft and including a mating element formed on a distal end thereof and adapted to mate with a fastening element to rotate the fastening element relative to a spinal connector coupled to the elongate shaft, thereby mating the fastening element to the connector. The connector-receiving member can be, for example, a housing having a recess formed therein for seating a spinal connector. The mating element on the driver shaft preferably extends through and distally beyond the housing, and in one embodiment, the mating element can be in the form of an elongate shaft. At least a portion of the shaft is preferably asymmetrical for extending into a socket formed in a fastending element. The shaft that forms the mating element can also include threads formed thereon for mating with corresponding threads formed on a spinal connector.

The present invention also provides a spinal fixation kit that includes a spinal clamp having top and bottom portions adapted to seat a spinal fixation element there between, and a fastening element having a head and a shaft. The top and bottom portions of the clamp member each include a bore extending therethrough and axially aligned with one another for receiving a fastening element, and the bore in the top portion includes left-handed threads formed therein. The threads are configured to mate with left-handed threads formed on at least a portion of the shaft of the fastening element. In a further embodiment, a distal-most end of the shaft of the fastening element can include a socket formed therein for receiving a driver tool. The kit can thus also include an installation device having a hollow elongate shaft with a distal, connector-receiving portion adapted to seat the connector, and a driver tool rotatably coupled to the shaft and including a distal end that is adapted to extend through the bores in the connector and into the socket in the fastening element such that rotation of the driver tool is effective to mate the fastening element to the connector. The kit can also include a spinal anchoring element having at least one thru-bore formed therein. The fastening element is preferably removably matable to the at least one thru-bore in the spinal anchoring element.

The present invention also provides a spinal fixation system that includes a spinal fixation plate having at least one thru-bore formed therein, and a spinal connector having top and bottom portions that are connected to one another at a terminal end thereof and that are adapted to retain a spinal rod there between. A locking mechanism can be provided for locking the top and bottom portions of the spinal connector in a fixed position with respect to one another to engage a spinal rod there between. The locking mechanism is also preferably adapted to mate the spinal connector to the spinal fixation plate.

In other aspects of the present invention, a medical connector device is provided in the form of a substantially J-shaped connector body having a first, substantially planar portion with a thru-bore formed therein, and a second, substantially curved portion that extends in a direction substantially transverse to the first portion and that defines a recess for seating a spinal fixation element. The thru-bore in the first portion is positioned to receive a fastening element such that a head of the fastening element is effective to contact a spinal fixation element seated within the recess in the second portion and to lock the spinal fixation element within the recess.

In yet another embodiment of the present invention, a spinal fixation system is provided having a spinal plate with at least one thru-bore formed therein, and at least one anchoring device having a distal hook portion adapted to engage bone and a proximal receiver head adapted to receive an elongate spinal fixation element. The receiver head is positionable within the at least one thru-bore formed in the spinal plate such that a spinal fixation element disposed within the receiver head can be coupled to the spinal plate, and the spinal plate can be mated to bone. In an exemplary embodiment, the spinal plate is an occipital plate having first and second opposed thru-bores formed therein, and the system includes a first anchoring device having a receiver head disposed within the first thru-bore formed in the occipital plate, and a second anchoring device having a receiver head disposed within the second thru-bore formed in the occipital plate. A locking mechanism can be disposed within the receiver head of the at least one anchoring device to lock a spinal fixation element therein.

### BRIEF DESCRIPTION OF THE DRAWINGS

The invention will be more fully understood from the following detailed description taken in conjunction with the accompanying drawings, in which:

FIG. 1A is perspective view of a clamp member for mating a spinal fixation element to a spinal fixation device in accordance with one embodiment of the present invention;

FIG. 1B is a side view of the clamp member shown in FIG. 1A;

FIG. 1C is a side cross-sectional view of the clamp member shown in FIG. 1A mated to a spinal fixation plate and having a spinal fixation rod extending therethrough;

FIG. 1D is a perspective view of the clamp member shown in FIG. 1A mated to a spinal fixation plate and having a spinal fixation rod extending therethrough;

FIG. 1E is a disassembled, perspective view of another embodiment of a connector clamp member and a fastening element for mating the clamp member to a spinal anchoring device in accordance with the present invention;

FIG. 1F is a side perspective view of an installation device for engaging a clamp member and positioning the clamp member relative to a spinal anchoring device in accordance with the present invention;

FIG. 1G is an enlarged view of a distal portion of the installation device shown in FIG. 1F;

FIG. 1H is a side perspective view of another embodiment of an installation device for engaging a clamp member and for mating a fastening element to the clamp member in accordance with the present invention;

FIG. 1I is a cross-sectional view of the installation device shown in FIG. 1H;

FIG. 1J is an enlarged view of the distal portion of the installation device shown in FIG. 1H;

FIG. 2A is a disassembled perspective view of a hinged clamp member for mating a spinal fixation element to a spinal fixation plate in accordance with another embodiment of the present invention;

FIG. 2B is a side perspective view of the hinged clamp member mated to the spinal fixation plate shown in FIG. 2A;

FIG. 3A is a side view of another embodiment of a hinged clamp member in accordance with the present invention integrally formed on a spinal fixation plate;

FIG. 3B is a top perspective view of the hinged clamp member and spinal fixation plate shown in FIG. 3A;

FIG. 4A is a top perspective view of a J-type connector mating a spinal fixation rod to a spinal fixation plate in accordance with yet another embodiment of the present invention;

FIG. 4B is a side cross-sectional view of the J-connector mated to the spinal fixation plate shown in FIG. 4A;

FIG. 5A is a side cross-sectional view of two spinal fixation hooks for mating spinal fixation elements to a spinal fixation plate that is coupled to the spinal fixation hooks, and that is attached to bone by the hooks in accordance with yet another embodiment of the present invention;

FIG. 5B is a disassembled view of the spinal fixation hooks and plate shown in FIG. 5A;

FIG. 5C is a perspective view of the spinal fixation hooks and plate shown in FIG. 5A;

FIG. 6A is a perspective view of an offset connector for mating a spinal fixation element to a spinal screw in accordance with another embodiment of the present invention;

FIG. 6B is a disassembled view of the offset connector and spinal screw shown in FIG. 6A;

FIG. 6C is a top view of the offset connector and spinal screw shown in FIG. 6A;

FIG. 6D is a bottom perspective view of the offset connector and spinal screw shown in FIG. 6A;

FIG. 7A is a perspective view of one embodiment of a receiver head for use with the offset connector shown in FIG. 6A;

FIG. 7B is a perspective view of another embodiment of a receiver head for use with the offset connector shown in FIG. 6A;

FIG. 7C is a perspective view of yet another embodiment of a receiver head for use with the offset connector shown in FIG. 6A;

FIG. 8A is a perspective view of another embodiment of an offset connector that is adapted to mate a spinal rod to a spinal screw in accordance with the present invention; and

FIG. 8B is a perspective view of the offset connector shown in FIG. 8A having a spinal rod positioned therein and mated to a spinal screw attached thereto.

### DETAILED DESCRIPTION OF THE INVENTION

The present invention provides various methods and devices for mating spinal fixation elements, such as spinal rods, to spinal anchoring devices, such as plates, hooks, bolts, wires, and screw. One of the many advantages of the connector devices is that they provide a low-profile connection between a spinal fixation element and spinal anchoring devices. Some of the connector devices also provide an adjustable connection, such that a position of the spinal fixation element with respect to the spinal anchoring device to which it is mated can be adjusted.

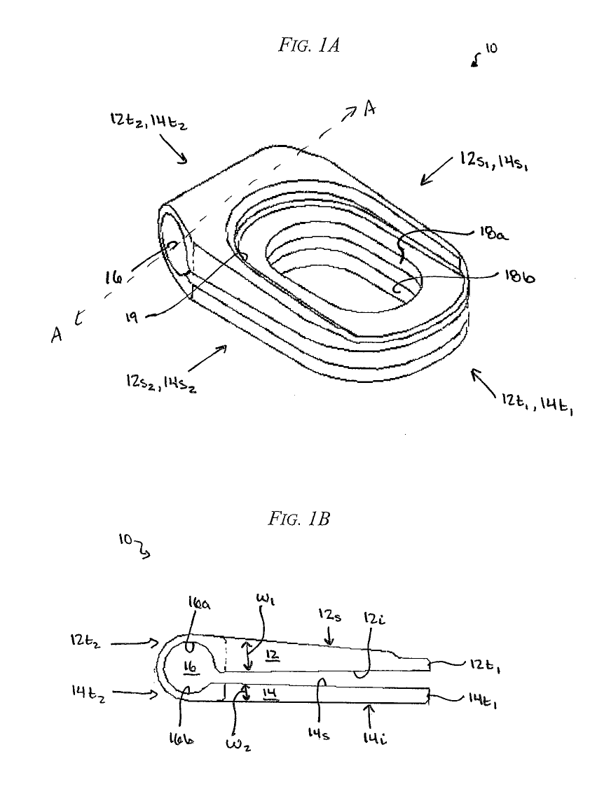

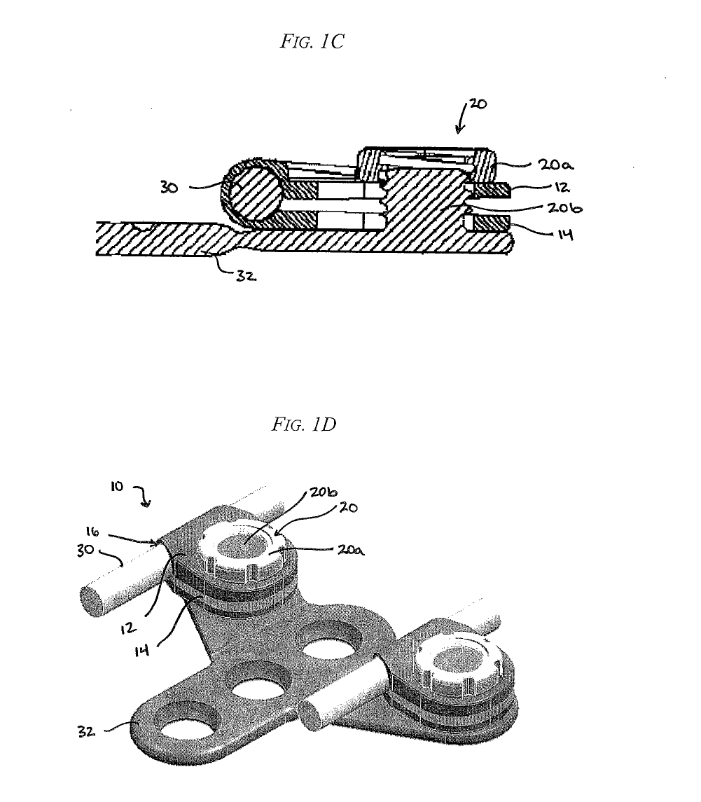

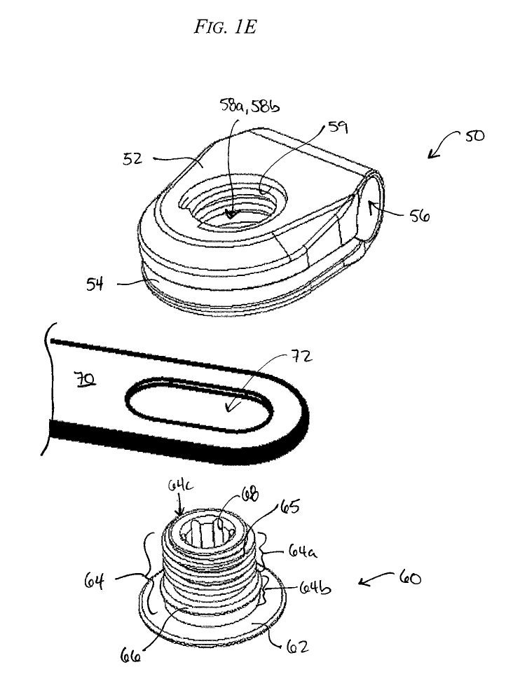

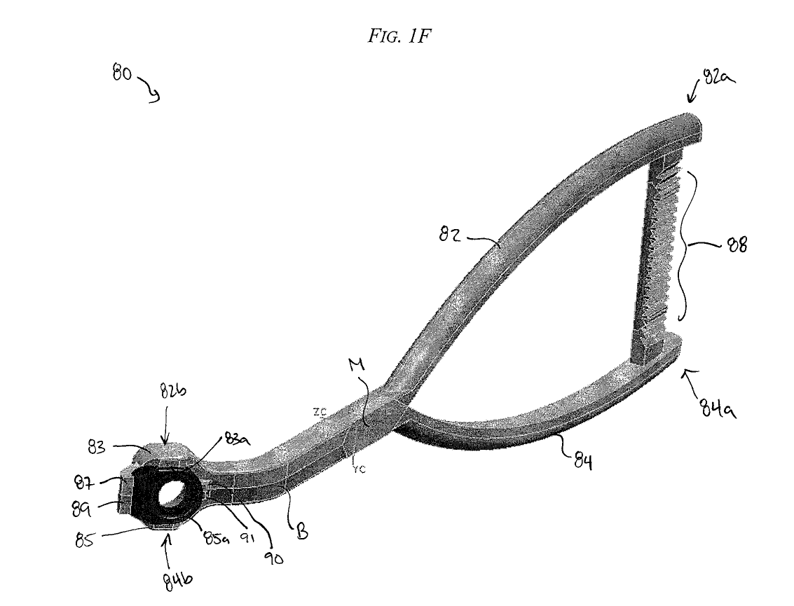

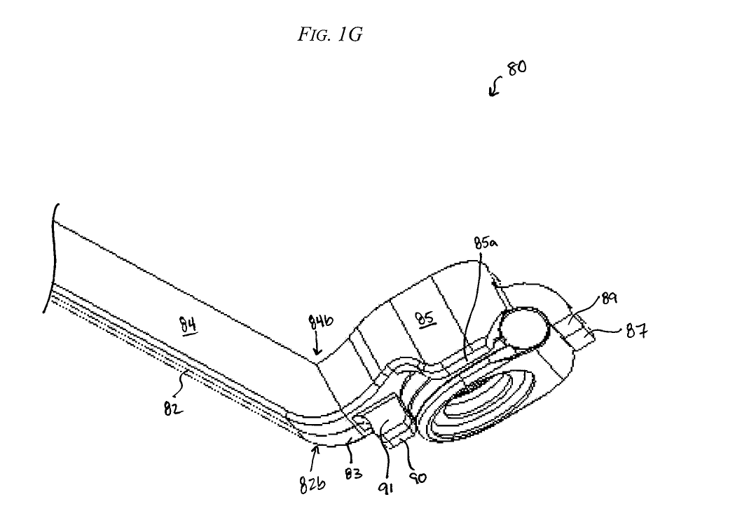

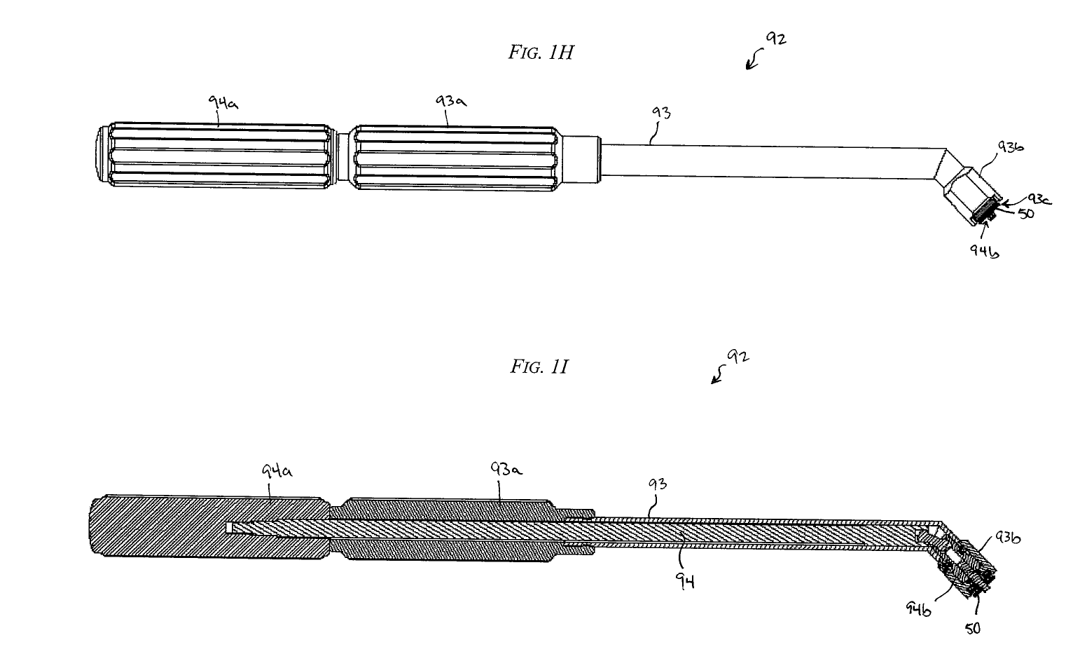

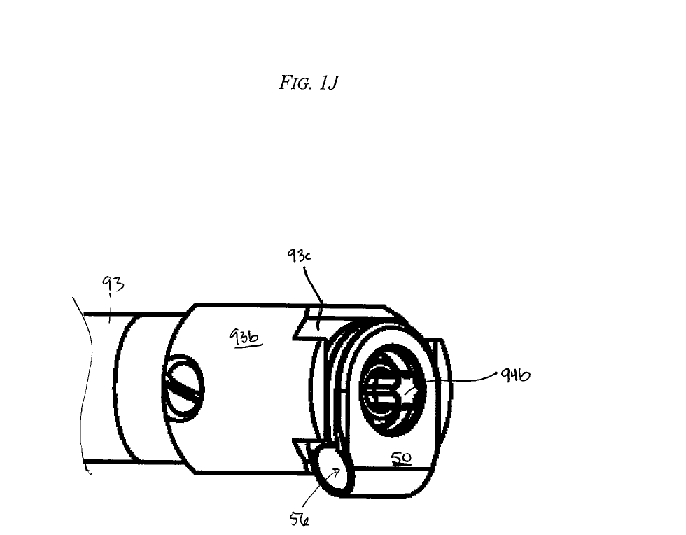

FIGS. 1A-1D illustrate one exemplary embodiment of a connector device 10 for mating a spinal fixation element to a spinal anchoring device. In this embodiment, the connector device 10 is in the form of a clamp member that is adapted to clamp a spinal fixation element, such as a spinal rod, there between, to mate the spinal fixation element to a spinal anchor, such as spinal plate. In general, the clamp 10 includes top and bottom portions 12, 14 that are connected to one another at a terminal end 12t₂, 14t₂ thereof such that the top and bottom portions 12, 14 are movable between an open position, in which the top and bottom portions 12, 14 are spaced a distance apart from one another, and a closed position, in which the portions 12, 14 are adapted to engage a spinal fixation element there between. The clamp 10 can also include a locking mechanism 20 (FIGS. 1C-1D) that is effective to lock the top and bottom portions 12, 14 in the closed position to retain the spinal fixation element there between. The locking mechanism 20 can also be adapted to lock the clamp 10 to a spinal anchoring device. While various fixation elements and anchoring devices can be used, FIGS. 1A-1D are described in connection with a spinal fixation rod 30 and a spinal plate 32.

The top and bottoms portions 12, 14 of the clamp 10 can have a variety of configurations, shapes and sizes, but they should be adapted to retain a spinal rod 30 there between, and to mate the rod 30 to a spinal plate 32. In an exemplary embodiment, as shown, each portion 12, 14 has a substantially elongate shape and includes superior and inferior surfaces 12s, 12i, 14s, 14i, first and second opposed sides 12s₁, 12s₂, 14s₁, 14s₂, and first and second opposed terminal ends 12t₁, 12t₂, 14t₁, 14t₂. The second terminal end 12t₂, 14t₂ of each portion is preferably mated to one another, or integrally formed with one another, such that the inferior surface 12i of the top portion 12 is positioned on the superior surface 14s of the bottom portion 14. The mated ends 12t₂, 14t₂ allow the top and bottom portions 12, 14 to be moved between an open position in which the portions 12, 14 are spaced apart from one another, as shown in FIGS. 1A-1B, and a closed position in which the portions 12, 14 are moved together to engage a spinal rod 30 disposed there between, as shown in FIGS. 1C-1D.

The top and bottom portions 12, 14 can optionally be biased to either a closed position or an open position such that a force greater than the biasing force is necessary to move the top and bottom portions 12, 14 from the closed position to the open position to insert a spinal rod there between, or from the open position to the closed position to engage a spinal rod positioned there between. In an exemplary embodiment, the top and bottom portions 12, 14 are biased to an open position, and they are formed from a material that allows the portions 12, 14 to deform around a spinal rod 30 positioned there between to provide a secure connection between the spinal rod 30 and the clamp 10. Suitable materials include, by way of non-limiting example, nitanol, titanium, and stainless steel. To facilitate deformation of the clamp 10 around a spinal rod 30, the inferior surface 12i of the top portion 12 and the superior surface 14s of the bottom portion 14 can tapered away from one another toward the first ends 12t₁, 14t₁ thereof, as shown in FIG. 1B. This allows the top and bottom portions 12, 14 to be compressed toward one another, to further provide a secure connection between the spinal rod 30 and the clamp 10. Other exemplary techniques for mating the top and bottom portions 12, 14 to one another will be discussed in more detail below.

In order to seat the spinal rod 30 between the top and bottom portions 12, 14, at least one recess is preferably formed on at least one of the inferior surface 12ᵢ of the top portion 12 and the superior surface 14ₛ of the bottom portion 14. While the position, shape, size, and configuration of the recess(es) can vary, in an exemplary embodiment, as shown, the recess 16 is formed in the second terminal ends 12t₂, 14t₂ of the top and bottom portions 12, 14. Such positioning of the recess 16 facilitates formation of a hinge-type connection between the top and bottom portions 12, 14, allowing the portions 12, 14 to be moved between the open and closed positions. The shape and size of the recess 16 can also vary, but it preferably defines a substantially cylindrical cavity extending between the opposed sides 12s₁, 12s₂, 14s₁, 14s₂ of the top and bottom portions 12, 14. The cylindrical cavity 16 can be formed from opposed concave recesses 16a, 16b formed in each of the inferior surface 12ᵢ of the top portion 12 and the superior surface 14ₛ of the bottom portion 14. The recess 16 preferably does not extend beyond the inferior surface 14ᵢ of the bottom portion 14 to allow the inferior surface 14ᵢ to have a substantially planar configuration. Such a configuration allows the inferior surface 14ᵢ of the clamp 10 to engage and rest against a spinal anchoring device, such as a spinal fixation plate, without interference from the spinal rod 30 disposed within the recess 16. In another embodiment, the recess 16 can have an elongate shape to allow the spinal rod 30 to be positioned at various locations within the recess 16, and to allow an angle of the spinal rod 30 with respect to an axis A of the recess 16 to be adjusted.

Once the spinal rod 30 is positioned within the recess 16 between the top and bottom portions 12, 14, the portions 12, 14 are preferably locked in the closed position using a locking mechanism 20. The locking mechanism 20 can also be used to attached the clamp 10 to a spinal plate 32. While a variety of techniques can be used to lock the clamp 10 in the closed position and to mate the clamp 10 to a spinal plate 32, in an exemplary embodiment, each portion 12, 14 includes a bore 18a, 18b formed therein for receiving a portion of the locking mechanism 20. The location of each bore 18a, 18b can vary depending on the intended use, and depending on the location of the spinal rod 30 within the clamp 10. In the illustrated embodiment, however, each bore 18a, 18b is formed in a substantial mid-portion of the clamp 10 to prevent the locking mechanism 20 from interfering with the spinal rod 30. The shape of the bores 18a, 18b can also vary, and in one embodiment the bores 18a, 18b can be substantially cylindrical to prevent sliding movement of a locking mechanism 20 therein. Alternatively, the bores 18a, 18b can be in the form of elongate slots 18a, 18b, as shown, which allow the clamp member 10 to slide with respect to the locking mechanism 20. As a result, when the clamp member 10 is mated to a spinal plate 32, the clamp member 10 can translate relative to the plate 32, thus allowing the position of a rod 30 engaged by the clamp member 10 to be adjusted with respect to the plate 32. One or both slots 18a, 18b can also optionally include a recess formed therein for seating a portion of the locking mechanism 20, which will be discussed in more detail below. As shown in FIG. 1A, a recess 19 is formed in the superior surface 12s of the top portion 12.

The locking mechanism 20 that is used to lock the clamp member 10 in the closed position, and to mate the clamp member 10 to a spinal plate 32, can also have a variety of configurations, and virtually any locking mechanism known in the art for use with spinal fixation devices, including polyaxial and monoaxial bone screws, hooks, plates, rods, etc., can be used. In the illustrated embodiment, however, the locking mechanism 20 includes a threaded member 20b that is integrally formed on and extends from a surface of the spinal fixation plate 32, and a locking nut 20a that is adapted to mate to the threaded member 20b. While not shown, the threaded member 20b can be separate from and disposed through a thru-bore formed in the spinal fixation plate 32. In use, the clamp member 10 is positioned with the threaded member 20b extending through the slot 18a, 18b in each of the top and bottom portions 12, 14 thereof, and the nut 20a is threaded onto the threaded member 20b to close the clamp member 10, thereby engaging the spinal rod 30 within the recess 16, and locking the clamp member 10 to the spinal plate 32.

A person skilled in the art will appreciate that a variety of other techniques can be used to lock the clamp member 10 in the closed position. By way of non-limiting example, the clamp member 10 can include gripping features and/or anti-rotation features formed within the recess 16 thereof to facilitate engagement of the spinal rod 30. In other embodiments of the invention, as shown in FIG. 1E, the locking mechanism can be adapted to mate with corresponding threads formed within a cylindrical bore formed in the top portion of the clamp member, rather than mating to a locking nut.

Referring to FIG. 1E, a clamp member 50 is shown that is similar to clamp member 10 shown in FIGS. 1A-1D, and that includes top and bottom portions 52, 54 that are mated to one another and that are configured to seat and retain a spinal fixation element within a recess 56 formed there between. The top and bottom portions 52, 54 also each include a bore 58a, 58b formed therethrough for receiving a locking mechanism, such as fastening element 60, that is adapted to lock the top and bottom portions 52, 54 in a fixed position related to one another to lock a spinal fixation element there between, and to lock the clamp 50 to a spinal anchoring device, such as plate 70. In this embodiment, the bore 58a formed through the top portion 52 of the clamp 50 includes threads 59 formed therein for mating with corresponding threads formed on the fastening element 60. The bore 58b in the bottom portion 54 of the clamp 50 is preferably adapted to allow free rotation of a locking mechanism extending therethrough.

The fastening element 60 includes a head 62 and a shaft 64. The head 62 has a size that prevents passage thereof through a thru-bore formed in a spinal anchoring device, such as thru-bore 72 formed in plate 70, and the shaft 64 is configured to extend through the thru-bore 72 in the plate 70 and through each of the top and bottom portions 52, 54 of the clamp 50. A distal portion 64a of the shaft 64 includes threads 65 formed thereon for mating with the corresponding threads 59 formed within the bore 58a in the top portion 52 of the clamp 50. Thus, in use, when the shaft 64 of the fastening element 60 is positioned through the thru-bore 72 in the plate 70 to threadably mate to the clamp 50, the fastening element 60 engages the top portion 52 of the clamp 50 to lock the top and bottom portions 52, 54 in a fixed position relative to one another, thereby locking a spinal fixation element there between and locking the clamp 50 to the plate 70.

Since the fastening element 60 is bottom loading relative to the plate 70, the fastening element preferably includes a mating element formed on a distal-most end 64c of the shaft 64 to allow the fastening element 60 to be engaged by a driver mechanism and rotated to thread the shaft 64 into the clamp 50. While a variety of mating elements can be used, in an exemplary embodiment, the mating element is an asymmetrical socket 68 that is adapted to receive a complementary asymmetrical driver mechanism. The illustrated socket 68 has a six-pointed star shape, but virtually any shaped socket can be used. In use, after the fastening element 60 is inserted through the thru-bore 72 in the plate 70, and the clamp 50 is positioned over the shaft 64, a driver mechanism is inserted into the socket 68 and it is rotated to thread the fastening element 60 into the clamp 50, thereby mating the clamp 50 to the plate 70.

In order to further facilitate mating of the fastening element 60 to the clamp 50, the fastening element 60 also preferably includes a feature that at least temporarily attaches the fastening element 60 to the plate 70. This will allow the fastening element 60 to be maintained in a fixed, but freely rotatable position relative to the plate 70 while a driver mechanism is used to rotate the fastening element 60 to threadably mate with the clamp 50. The fastening element 60 can also optionally be freely translatable relative to the plate 70, depending on the configuration of the thru-bore 72 in the plate 70. While various techniques can be used to at least temporarily mate the fastening element 60 to the plate 70, as shown in FIG. 1E the proximal portion 64a of the shaft 64 can include a flange 66 formed there around. The flange 66 is adapted to pass through the thru-bore 72 in the plate 70 such that the fastening element 60 mates to the plate 70 using a snap-on connection. As a result, the fastening element 60 will not separate from the plate 70 when the plate is being implanted, and when the fastening element 60 is being mated to the clamp 50. Thus, the clamp 50 can be placed over the shaft 64 of the fastening element 60 and a driver mechanism can be inserted into the socket 68 to thread the fastening element 60 to the clamp 50. In an exemplary embodiment, the threads 59, 68 formed on the clamp 50 and the fastening element 60 are left-handed threads. This allows a driver mechanism to be rotated in a right-handed direction when the device is being implanted. Further, the threads 59, 68 can also be square threads to aid in ease of application of the clamp 50 to the fastening element 60, and to reduce the risk of cross threading.

The present invention also provides an installation device 80, shown in FIGS. 1F-1G, that is adapted to engage the clamp 50 to allow the clamp 50 to be positioned over the shaft 64 of the fastening element 60 and to be maintained in that position while the fastening element 60 is being mated to the clamp 50. The installation device 80 can also be used to position a spinal fixation element, such as a spinal rod, within the recess 56 in the clamp 50, e.g., by sliding the clamp 50 over the rod.

In general, the installation device 80 includes first and second opposed arms 82, 84, each having a proximal handle 82a, 84a and distal, clamp-engaging end 82b, 84b. The arms 82, 84 are pivotally mated to one another such that the arms 82, 84 are movable between an open position, in which the distal ends 82b, 84 are spaced a distance apart from one another, and a closed position, as shown, in which the arms 82, 84 are effective to engage and retain the clamp 50 there between. In the illustrated embodiment, the arms 82, 84 are mated to one another at a substantial midpoint M thereof, such that the arms 82, 84 pivot about the midpoint M. A locking mechanism, such as the illustrated pawl-and-ratchet mechanism 88, can be coupled to the arms 82, 84 to lock the arms 82, 84 in the closed position. A person skilled in the art will appreciate that virtually any type of locking mechanism can be used. The arms 82, 84 can also include at least one angle or bend B formed therein to facilitate use of the device, as will be described in more detail below.

FIG. 1G illustrates the distal end 82b, 84b of the arms 82, 84 in more detail (only a portion of arm 82 is shown), and as shown, the distal end 82b, 84b of each arm 82, 84 includes a clamp-engaging member 83, 85 formed thereon. Each member 83, 85 is substantially C-shaped such that when the arms 82, 84 are in the closed position, the members 83, 85 define a substantially cylindrical recess formed there between for seating the clamp 50. Each member 83, 85 also preferably includes an engagement mechanism formed thereon and adapted to engage the clamp 50 to prevent the clamp 50 from becoming disengaged from the installation device 80. While the engagement mechanism can have a variety of configurations, in FIGS. 1F-1G, each clamp-engaging member 83, 85 includes a ridge 83a, 85a formed thereon and positioned to extend between the top and bottom portions 52, 54 of the clamp 50 when the arms 82, 84 are in the closed position. The clamp-engaging members 83, 85 can also include features to facilitate alignment of the installation device 80 with the clamp 50. For example, each member 83, 85 can include an extension tab 87, 89 formed on a distal-most end thereof that has a shape that complements the shape of the rod-receiving portion of the clamp 50 such that the tabs 87, 89 extend around a portion of the clamp 50. A second set of tabs 90, 91 opposed to the first set of tabs 87, 89 can also be formed on the arms 82, 84 to further align the clamp 50 relative to the clamp-engaging members 83, 85.

In use, the installation device 80 can engage the clamp 50 between the clamp-engaging member 83, 85 on each arm 82, 84. With the arms 82, 84 preferably in a locked position, using the pawl-and-ratchet mechanism 88, the installation device 50 can be manipulated to slide the clamp 50 over a spinal rod (not shown), and then to position the clamp 50 over the shaft 64 of the fastening element 60 (FIG. 1E). A driver mechanism (not shown) can then be inserted into the socket 68 in the fastening element 60 to thread the fastening element 60 into the clamp 50, thereby securely attaching the clamp 50 to the plate 70 and engaging a spinal rod within the recess 56 in the clamp 50.

FIGS. 1H-1J illustrate another embodiment of an installation tool for use with the clamp 50 shown in FIGS. 1E-1F. In this embodiment, the installation tool 92 is adapted to engage the clamp 50 and to rotate the fastening element 60 to mate the fastening element 60 to the clamp 50. In particular, the installation tool 92 includes a hollow elongate shaft 93 having a proximal gripping portion 93a and a distal clamp-engaging portion 93b. The clamp-engaging portion 93b can optionally be rotatably coupled to the shaft 93, or it can be positioned at a fixed angle relative to the shaft 93, as shown, to facilitate use of the device. In particular, the angled shaft 93 prevents the shaft 93 from interfering with visual access to the clamp-engaging portion 93b, and the implant site for the clamp 50 mated thereto. The clamp-engaging portion 93b is preferably in the form of a housing having a recess 93c formed in a distal-end thereof for seating the clamp 50. The recess 93c preferably has a shape that is complementary to the shape of the clamp 50 such that the clamp 50 will be substantially aligned with a driver shaft 94 on the installation tool 92.

The driver shaft 94 is rotatably disposed through the hollow elongate shaft 93 and it includes a proximal handle 94a and a distal mating element 94b formed thereon. The distal mating element 94b is configured to extend through the clamp 50 positioned within the recess 93c of the clamp-engaging portion 93b, and to extend into the socket 68 in the fastening element 60. The mating element 94b therefore preferably has a shape that is complementary to the shape of the socket 68 such that the mating element 94b can be used to rotate the fastening element 60, thereby threading the fastening element 60 into the clamp 50. Rotation of the mating element 94b on the driver shaft 94 can be achieved by rotating the handle 94a of the shaft 94, which is connected to the mating element 94b.

The mating element 94b on the driver shaft 94 can also optionally be threaded to mate with the threads 59 formed within the bores 58a, 58b in the clamp 50. This will allow the mating element 94b to engage the clamp 50 and retain the clamp 50 within the recess 93c prior to mating the clamp 50 to the plate 70 using the fastening element 60. Thus, in use, the clamp 50 can be loaded onto the installation tool 92 by positioning the mating element 94b on the driver shaft 94 through the bores 58a, 58b in the clamp 50. The handle 94a on the driver shaft 94 can then be rotated, preferably while holding the gripping portion 93a of the hollow shaft 93, to thread the mating element 94b into the bores 58a, 58b in the clamp 50. The installation tool 92 can then be manipulated to slide the clamp 50 over a spinal fixation element, e.g., a rod, by inserting the rod through the recess 56 (FIG. 1J) in the clamp 50. The tool 92 can then be further manipulated to position the clamp 50 over the shaft 64 of the fastening element 60 such that the mating element 94b on the driver shaft 94 extends into the socket 68 in the fastening element 60. The handle 94s can then be rotated to rotate the mating element 94b, thereby rotating the fastening element 60 to mate the fastening element 60 to the clamp 50. A person skilled in the art will appreciate that a variety of other techniques can be used to engage the clamp 50 and/or to rotating the fastening element 60 to mate it to the clamp 50.

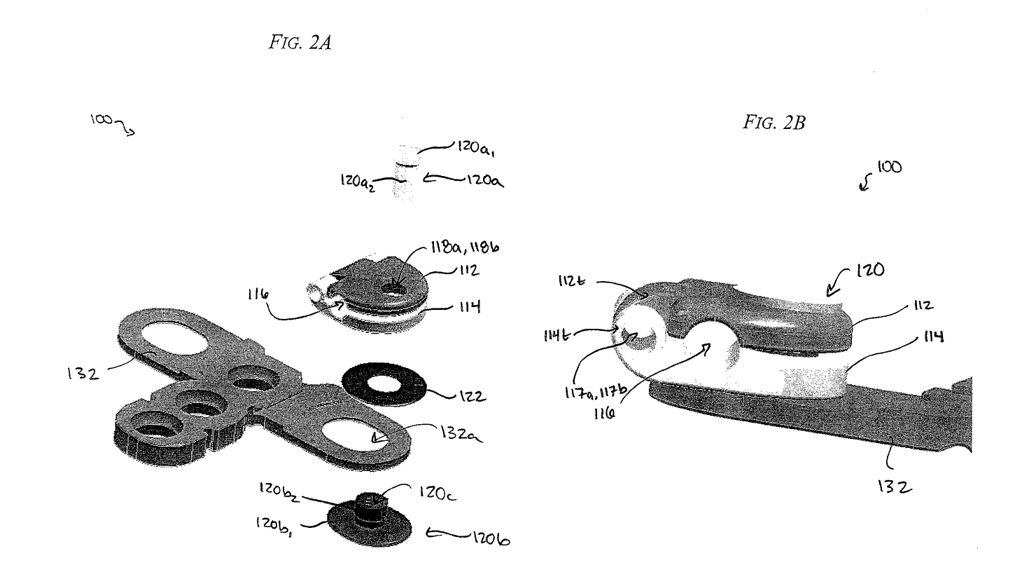

FIGS. 2A-2B illustrate yet another embodiment of a connector device 100. In this embodiment, the connector device 100 is in the form of a hinged clamp. The hinged clamp 100 is similar to clamp 10 in that it includes top and bottom portions 112, 114 that are mated to one another and movable between an open position in which the portions 112, 114 are spaced apart from one another, and a closed position in which the portions 112, 114 are adapted to engage a spinal rod (not shown) there between. The hinged clamp 100 also similarly includes a recess 116 formed in the top and bottom portions 112, 114 for receiving a spinal rod. The clamp 100 differs from the clamp 10, however, in that it includes a hinged connection formed between the top and bottom portions 112, 114. In particular, the top portion 112 includes a terminal end 112t that is in the form of a female component, and the bottom portion 114 includes a terminal end 114t that is in the form of a male component for receiving the female component on the top portion 112. Each portion 112, 114 also includes a pathway 117a, 117b extending through. When the pathway 117a, 117b in each portion 112, 114 is axially align with one another, a pin member (not shown) can be disposed therethrough for pivotally mating the top and bottom portions 112, 114 to one another. In use, the top and bottom portions 112, 114 can pivot about the pin member to move between the open and closed positions, thus allowing a spinal rod to be positioned and engaged there between. One skilled in the art will appreciate that the pin member can be integrally formed with either the top portion 112 or the bottom portion 114 of the clamp 100. Moreover, the top and bottom portions 112, 114 can optionally be adapted to snap-fit together.

As previously described with respect to clamp member 10, a locking mechanism can be used to lock the top and bottom portions 112, 114 in a fixed position with respect to one another. By way of non-limiting example, FIG. 2A illustrates another embodiment of a locking mechanism 120 that includes a threaded member 120a and a receiving element 120b. The threaded member 120a generally includes a head 120a₁ and a threaded shank 120a₂, and the receiving element 120b generally includes a head 120b₁ and a shank 120b₂ having a threaded bore 120c formed therein. In use, the threaded member 120a and the receiving element 120b are positioned through opposed sides of a bore 118a, 118b formed in each of the top and bottom portions 112, 114. The threaded member 120a is then threaded into the threaded bore 120c formed in the receiving element 120b to lock the spinal plate 132 and the top and bottom portions 112, 114 of the clamp 100 there between. The threaded member 120a and the receiving element 120b are also effective to bring the top and bottom portions 112, 114 toward one another to the closed position, thereby engaging a spinal rod there between and preventing translation and rotation of the clamp 100 relative to the plate 132. Since the spinal fixation plate 132 is preferably attached to bone prior to locking the clamp 100 in a closed position, the retaining element 120b is preferably pre-loaded through the thru-bore 132a formed in the plate 132, and also preferably through the bores 118a, 118b formed in the clamp 100. A washer 122, as shown, can optionally be used to engage a portion of the retaining element 120b to prevent removal of the retaining element 120b from the plate 132. Alternatively, the bottom portion 114 of the clamp 100 can be adapted to engage a portion of the retaining element 120b to prevent removal of the retaining element 120b from the plate 132 and from the clamp 100.

As previously stated with respect to FIGS. 1A-1D, a person skilled in the art will appreciate that a variety of other techniques can be used to lock the clamp 100 in a closed position and to lock the clamp 100 to a spinal plate.

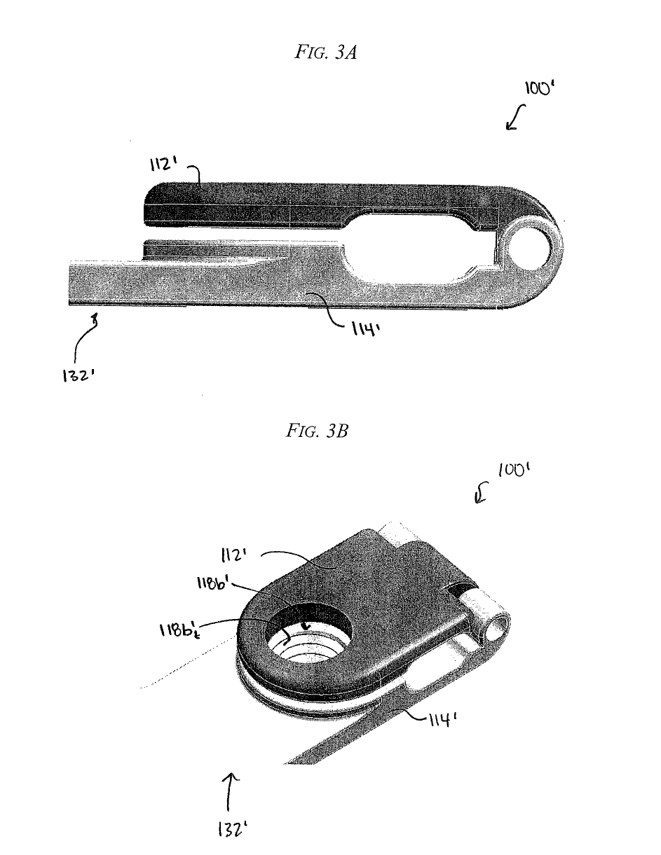

FIGS. 3A-3B illustrate yet another embodiment of the present invention, in which a connector member 100′ is formed integrally with a spinal fixation plate 132′. As shown, the connector member 100′ is similar to the hinged clamp 100 shown in FIGS. 2A-2B, however the bottom portion 114′ of the clamp 100′ is formed integrally with the spinal fixation plate 132′. As a result, the top portion 112′ is hingedly connected to the plate 132′ and it is movable between an open position and a closed position. FIGS. 3A-3B also illustrate another embodiment of a technique for locking the clamp 100′ in the closed position. As shown, the bottom portion 114′ of the clamp 100′ includes threads 118bt′ formed within the bore 118b′ extending therethrough for directly mating with a threaded member, such as a screw (not shown).

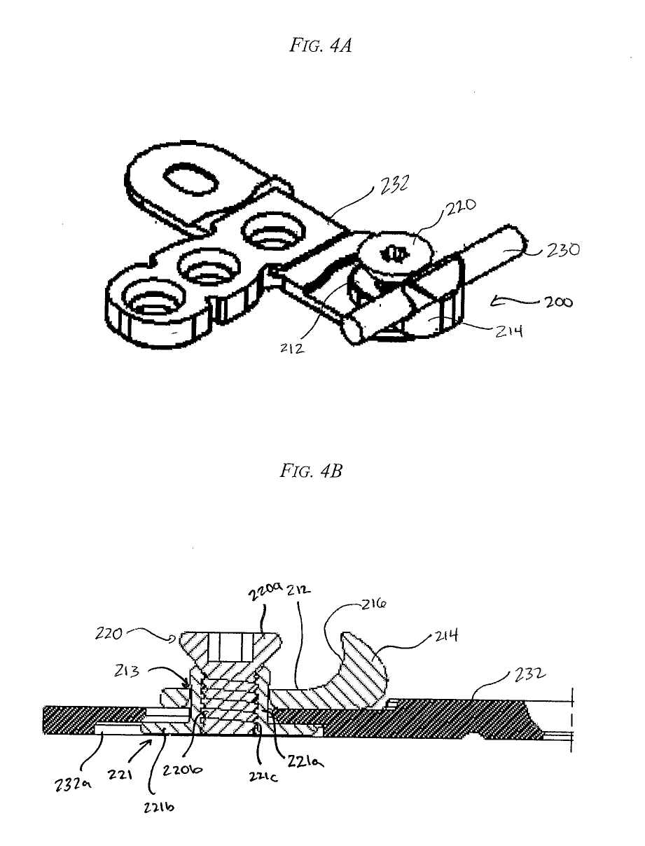

FIGS. 4A-4B illustrate yet another embodiment of a connector member 200 in accordance with the present invention. In this embodiment, the connector member 200 is substantially J-shaped and it includes a first, substantially planar portion 212, and a second, substantially curved portion 214 that extends in a direction substantially transverse to the first portion 212. The curved portion 214 defines a recess 216 formed on an inner surface thereof for seating a spinal fixation element, such as spinal rod 230, as shown. The connector member 200 also includes a thru-bore 213 formed in the first portion 212 for receiving a portion of a locking mechanism.

The thru-bore 213 is preferably positioned relative to the curved portion 214 such that a portion of the locking mechanism, which will be discussed in more detail below, comes into contact with the spinal rod 230 seated within the recess 216. As a result, when the locking mechanism is in a locked configuration with the connector member 200, the locking mechanism will create an interference or friction fit with the spinal rod 230, thereby locking the spinal rod 230 within the recess 216.

While a variety of locking mechanism can be used, in the embodiment shown in FIGS. 4A-4B, the locking mechanism includes a fastening element 220 having a head 220a and a threaded shank 220b, and a receiving element 221 having a cylindrical portion 221a with a threaded bore 221c, and a flange 221b formed around a terminal end thereof. In use, the receiving element 221 is inserted up through the bottom of a spinal fixation device, such as plate 232 as shown, and up through the connector member 200. The flange 221b can be positioned against a surface of the spinal plate 232, or it can be positioned within a corresponding recess 232a formed in the spinal plate 232, as shown. Once the spinal rod 230 is positioned in the recess 216 in the curved portion 214 of the connector member 200, the shank 220b of the fastening element 220 can be inserted into the cylindrical portion 221a of the receiving element 221 to mate with the threads 221b formed therein. A loose mating connection between the threaded member 220 and the receiving element 221 will allow the spinal rod 230 to be slidably adjusted with respect to the connector member 200. Where the plate 232 or the connector 200 includes a slotted thru-bore formed therein, such a loose mating connection can also allow the connector member 200 to be adjusted with respect to the spinal plate 232. Once the spinal rod 230 is positioned as desired, the threaded member 220 can be fully threaded into the receiving element 221 to provide a locking connection there between, thereby locking the spinal rod 230 within the recess 216, and locking the connector 200 in a fixed position relative to the spinal fixation plate 232. A person skilled in the art will appreciate that a variety of other fastening techniques can be used to mate the connector member 200 and a spinal rod 230 to a spinal fixation plate 232, or to another spinal anchoring device.

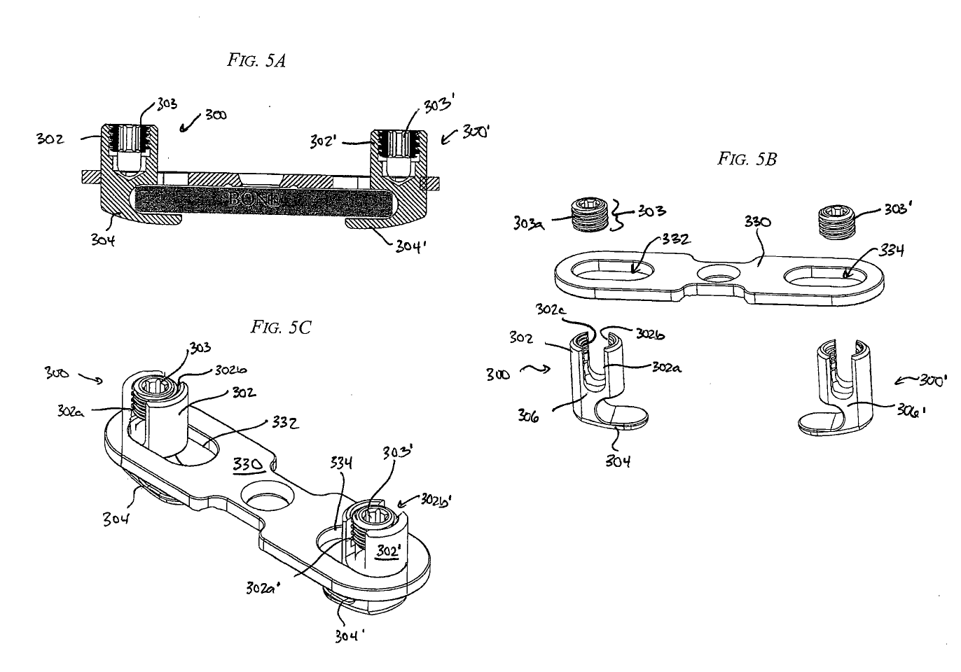

FIGS. 5A-5C illustrate yet another exemplary embodiment of the present invention. In this embodiment, the connector member 300 is in the form of a spinal hook having a proximal receiving member 302 for seating a spinal fixation element, such as a spinal rod (not shown), and a distal bone-engaging portion that is in the form of a hook 304. The receiving member 302 has a substantially cylindrical shape and it includes opposed U-shaped slots 302a, 302b formed therein and extending distally from the proximal end thereof. The slots 302a, 302b are configured to seat a spinal fixation element, such as a spinal rod. The receiving member 302 is also adapted to receive a closure mechanism or fastening element 303 for locking a spinal rod therein. In an exemplary embodiment, the closure mechanism 303 includes threads 303a formed thereon for mating with corresponding threads 302c formed within the receiving member 302. A person skilled in the art will appreciate that a variety of other mating techniques can be used to lock a spinal fixation element, such as a rod, within the receiving member 302 of connector member 300.

In use, two connector members 300, 300′ are preferably used to mate two spinal rods to a spinal plate 330 having at least two thru-bores or slots 332, 334 formed therein, as shown. The receiving member 302, 302′ on each connector member 300, 300′ is preferably configured to be position within a slot 332, 334 formed in the spinal plate 230 to mate two spinal rods (not shown) to the plate 230, and the hook portion 304, 304′ on each connector member 300, 300′ is used to engage bone. Depending on the configuration of the thru-bores 332, 334 in the plate 330, each receiving member 300, 300′ can optionally be adapted to allow rotatable and/or slidable movement thereof with respect to the plate 330. For example, in the illustrated embodiments, the plate 300 includes elongate slots 332, 334 formed therein, and the receiving member 302, 302′ on each connector member 300, 300′ includes opposed flattened side surfaces 306, 306′ (only one side is shown) formed thereon. The flattened sides 306, 306′ prevent rotation of the receiving members 302, 302′ within the slots 332, 334, yet they allow slidable movement of the receiving members 302, 302′ therein. This allows a spinal rod coupled to the receiving member 302, 302′ of each connector 300, 300′ to be positioned as desired with respect to the spinal plate 330. Once a rod is properly positioned relative to each connector 300, 300′, a closure mechanism 303, 303′ can be threaded into the receiver members 302, 302′ to lock the rods therein. As a result, when the closure mechanism 303, 303′ bears against the rods to lock the rods within the receiving members 302, 302′, the rods also preferably bear against the spinal fixation plate 300, thereby locking the connector members 300, 300′ to the plate 330. A person skilled in the art will appreciate that the connector members 300, 300′ can have a variety of configurations. By way of non-limiting example, the plate 330 can be telescoping and each connector member 300, 300′ can be integrally formed on opposed ends of the plate 330.

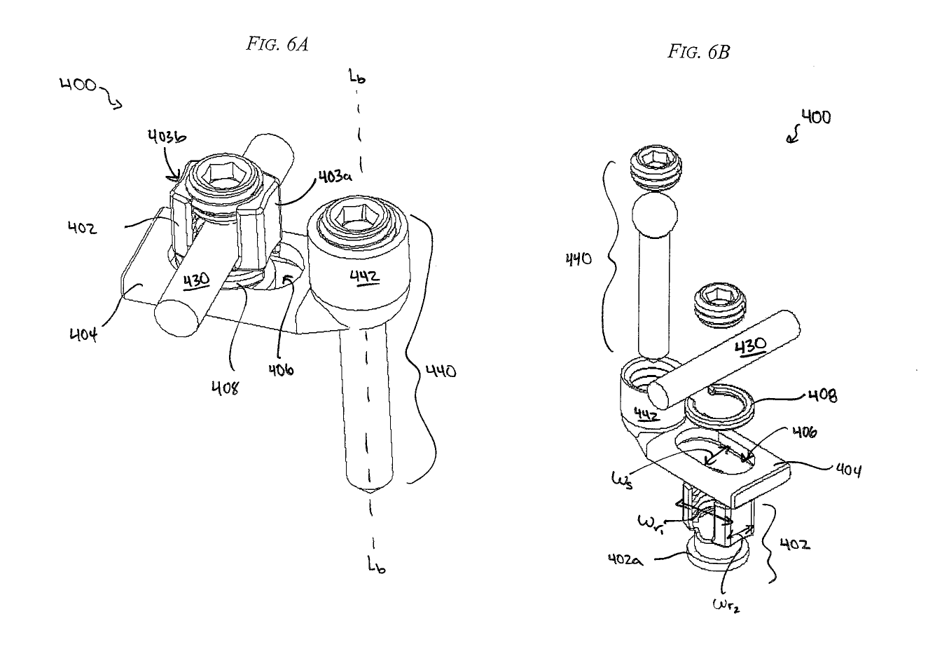

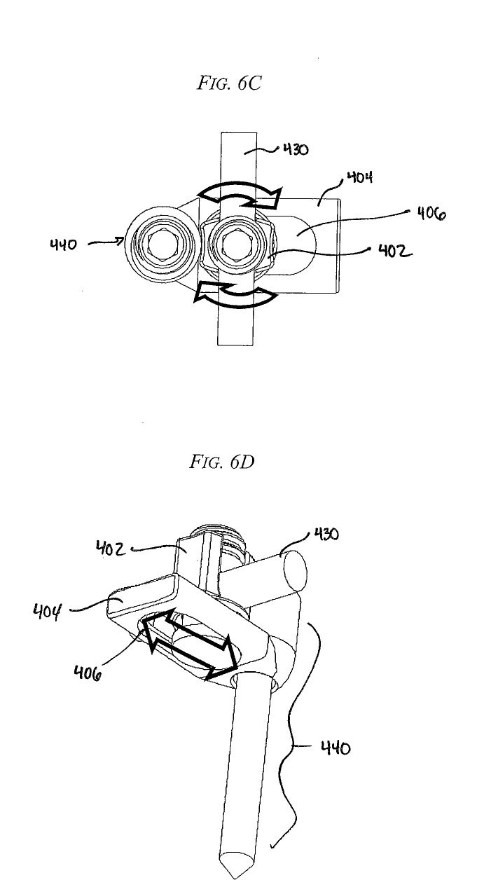

FIGS. 6A-6D illustrate yet another embodiment of a connector member 400 in accordance with the present invention. In this embodiment, the connector member 400 is an offset connector that is designed to mate a spinal fixation element, e.g., rod 430, to a spinal anchoring device, e.g., bone screw 440, such that the rod 430 is positioned offset from a longitudinal axis L_{b} of the bone screw 440. In particular, the connector member 400 includes a receiving head 402 that is adapted to seat a spinal rod 430, and that is adapted to be disposed through a thru-bore or slot 406 formed in a lateral extension member 404. The lateral extension member 404, in turn, is formed on or mated to a spinal anchoring device. While any spinal anchoring device can be used, in the illustrated embodiment the spinal 6anchoring device is a polyaxial screw 440. The receiving head 402 is similar to the receiving head 302 described about with respect to FIGS. 5A-5C, but it does not include a distal hook portion. Rather, the distal end of the receiving head 402 is in the form of a flange 402a that is sized to prevent passage thereof through the thru-bore or slot 406 formed in the lateral extension 404.

In use, the receiving head 402 can be positioned through the thru-bore or slot 406 formed in the lateral extension 404, and a spinal rod 430 locked in the receiving head 402 can be used to prevent removal of the receiving head 402 from the slot 406. A retaining member, such as a split o-ring 408, can optionally be disposed around a portion of the receiving head 402 on an opposed side of the slot 406 from the flange 402a to help retain the receiving head 402 within the slot prior to locking the rod 430 therein. The receiving head 402 can also be configured to prevent passage thereof through the slot 406 when positioned in a first orientation, and to allow passage through the slot 406 in the lateral extension 404 when position in a second orientation. This can be achieved by providing opposed, substantially flattened sides 403a, 403b on the receiver head 402 that define a width wᵣ₂ that is less than a width w of the slot 406 to allow the receiving head 402 to pass through the slot 406 in the second orientation, as shown in FIG. 6B. A width w of the receiving head 402 extending in a direction transverse to width w can be greater than the width w of the slot 406 to prevent passage of the receiving head 402 through the slot 406 in the first orientation (not shown).

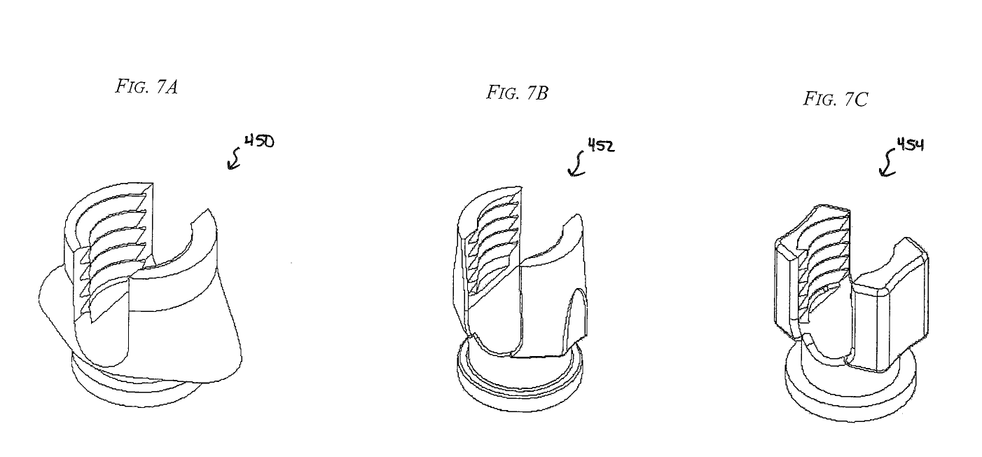

In another embodiment, opposed sides 403a, 403b on the receiver head 402 can be adapted to rest against the head portion 442 of the spinal screw 440 that is attached to the lateral extension 404. For example, the sides 403a, 403b can be substantially concave, as shown in FIGS. 6A-6C, to rest against the concave outer surface of the head portion 442 of the spinal screw 440. This is particularly useful where the receiver head 402 is slidably disposed within a slot 406 formed in the lateral extension 404 to allow lateral adjustment of the spinal rod 430 with respect to the spinal screw 440. Various alternative embodiments of a receiver head 450, 452, 454 are shown in FIGS. 7A-7C.

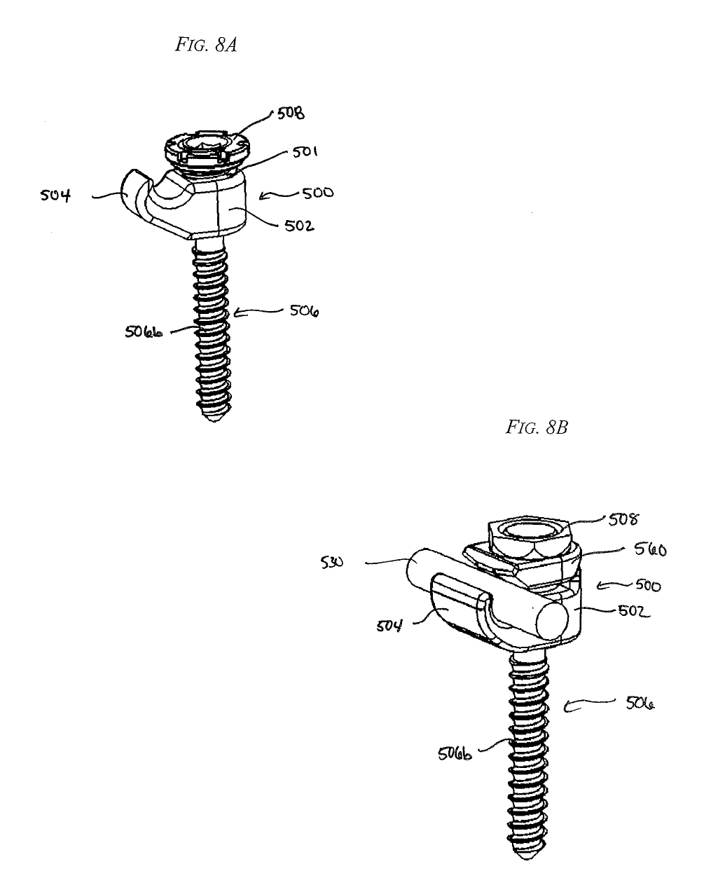

FIGS. 8A-8B illustrate yet another embodiment of a connector member 500. The connector member 500 is similar to the J-shaped connector 200 shown in FIGS. 4A-4B, but it is adapted to connect to a spinal screw, rather than a plate. In particular, the connector member 500 includes a first portion 502 having a bone screw 506 coupled thereto, and a second, J-shaped portion 504 that is adapted to seat a spinal rod therein. The first portion 502 preferably includes a bore 501 extending therethrough for receiving a shank 506b, and for seating a head (not shown) of the bone screw 506. In an exemplary embodiment, the bone screw 506 is a polyaxial bone screw, and the head of the screw 506 sits within a receiving recess formed in the bore 501 that extends through the first portion 502 of the connector 500.

In use, as shown in FIG. 8B, a locking mechanism, e.g., locking nut 508, can be applied to the first portion 502 of the connector member 500, either by attaching it to a portion of the bone screw 506, or more preferably by attaching it directly to the first portion 502. In an exemplary embodiment, the locking nut 508 is threaded onto a threaded member formed on the first portion 502. The locking nut 508, when fully threaded, can abut a spinal rod 530 to lock the rod 530 within the J-shaped portion that forms the second portion 504, thereby attaching the spinal rod 530 to the spinal screw 506. In the illustrated embodiment, however, a wedge-shaped member 560 is coupled to the first portion 502 of the connector 500, and it is effective to abut the spinal rod 530 to maintain the rod 530 within the J-shaped portion by an interference fit. The wedge-shaped member 560 is positioned between the locking nut 508 and the first portion 502 of the connector 500 such that the locking nut 508 can be used to lock the wedge-shaped member 560, and thus the spinal rod 530, in a fixed position. When the screw 506 is a polyaxial screw, a first locking element can be used to lock the polyaxial screw in a fixed orientation relative to the connector 500, and a second locking element can be used to lock the rod 530 to the connector 500. The element used to lock the polyaxial screw in a fixed orientation is preferably an inner set screw (not shown) that is applied to the bore 501 through which the screw 506 is placed, and the element used to lock the rod 530 to the connector 500 is preferably outer locking nut 508, which can thread onto the inner set screw to lock the rod 530 to the connector 500. A person skilled in the art will appreciate that a variety of other techniques can be used to engage the spinal rod 530 and lock the rod 530 in a fixed position with respect to the connector 500.

One of ordinary skill in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

1. An implantable spinal connector for mating a spinal fixation element to a spinal anchoring device, comprising:
a clamp member having top and bottom portions that are connected to one another at a terminal end thereof such that the top and bottom portions are movable between an open position in which the top and bottom portions are spaced a distance apart from one another, and a closed position in which the clamp member is adapted to engage a spinal fixation element there between, the clamp member further including a bore extending through the top and bottom portions for receiving a locking mechanism for locking the top and bottom portions in the closed position, the bore in at least one of the top and bottom portions being internally threaded for mating with corresponding threads formed on at least a potion of the locking mechanism.

2. The implantable spinal connector of claim 1, further comprising a recess formed between the top and bottom portions for seating a spinal fixation element.

3. The implantable spinal connector of claim 2, wherein the recess is formed adjacent to said terminal end for seating a spinal fixation element therein.

4. The implantable spinal connector of claim 2, wherein the recess is formed in at least one of the inferior surface of the top portion and the superior surface of the bottom portion.

5. The implantable spinal connector of claim 4, wherein the recess is formed in each of the inferior surface of the top portion and the superior surface of the bottom portion of the clamp member.

6. The implantable spinal connector of claim 5, wherein each recess has a concave shape such that the recesses together define a substantially cylindrical recess when the clamp member is in the closed position.

7. The implantable spinal connector of claim 1, wherein the top and bottom portions are hingedly coupled to one another at the terminal end thereof.

8. The implantable spinal connector of claim 1, further comprising a pivot pin extending through the terminal end of each of the top and bottom portions for hingedly mating the top and bottom portions to one another.

9. The implantable spinal connector of claim 8, wherein the pivot pin extends through a bore formed through and extending along a terminal end of each of the top and bottom portions of the clamp member.

10. The implantable spinal connector of claim 9, further comprising a recess formed between the top and bottom portions for receiving a spinal fixation element, the recess extending in a direction substantially parallel to a direction of the bore formed through and extending along a terminal end of each of the top and bottom portions of the clamp member.

11. The implantable spinal connector of claim 1, wherein the top and bottom portions are biased to a closed position such that a force greater than the biasing force must be applied to move the top and bottom portions to the open position.

12. The implantable spinal connector of claim 1, wherein the top and bottom portions are biased to an open position such that a force greater than the biasing force must be applied to move the top and bottom portions to the closed position.

13. The implantable spinal connector of claim 1, further comprising a locking mechanism disposable through the bore and effective to lock the top and bottom portions in the closed position to retain a spinal fixation element there between.

14. The implantable spinal connector of claim 13, wherein the locking mechanism comprises a fastening element having a head and a shaft, and wherein one of the bore formed in the top portion and the bore formed in the bottom portion of the clamp member is adapted to freely rotatably receive the threaded shaft of the fastening element, and the other one of the bore formed in the top portion and the bore formed in the bottom portion is internally threaded to mate to threads formed on at least a portion of the shaft of the fastening element.

15. The implantable spinal connector of claim 14, wherein fastening element includes a flange formed there around and adapted to at least temporarily mate the fastening element to a spinal anchoring device.

16. The implantable spinal connector of claim 14, wherein the bore in the top portion of the clamp member is internally threaded for mating with corresponding threads formed on at least a portion of the shaft.

17. The implantable spinal connector of claim 16, wherein the threads in the bore in the top portion of the clamp member and the threads formed on at least a portion of the shaft are left-handed threads.

18. The implantable spinal connector of claim 16, wherein the fastening element includes a mating element formed on a distal-most end thereof for mating with a driver tool.

19. The implantable spinal connector of claim 18, wherein the mating element comprises a socket.

20. The implantable spinal connector of claim 1, wherein the bottom portion of the clamp member is formed integrally with a spinal fixation plate.

21. The implantable spinal connector of claim 1, further comprising a recess formed in a superior surface of the top portion of the clamp member for seating a head of a fastening element.

22. The implantable spinal connector of claim 1, wherein the clamp member is formed from a material that allows the clamp member to deform around a spinal fixation element disposed between the top and bottom portions when the clamp member is locked in the closed position.

23. An implantable spinal connector for mating a spinal fixation element to a spinal anchoring device, comprising:
a clamp member having top and bottom portions that are connected to one another at a terminal end thereof such that the top and bottom portions are movable between an open position and a closed position;
a recess formed between a superior surface of the top portion of the clamp member and an inferior surface of the bottom portion of the clamp member, the recess being adapted to seat a spinal fixation element therein;
axially aligned, concentric bores extending through the top and bottom portions at a location spaced apart from the recess, the bores being configured to receive a locking mechanism for locking the top and bottom portions in the closed position; and
a substantially planer inferior surface extending along the bottom portion of the clamp member and configured to engage a spinal fixation plate.

24. The implantable spinal connector of claim 23, wherein at least one of the concentric bores includes threads formed therein.

25. The implantable spinal connector of claim 24, wherein the threads are left-handed threads.

26. An implantable spinal connector, comprising:
top and bottom portions hingedly mated to one another at a terminal end thereof, and having a recess formed there between for seating a spinal rod, the top and bottom portions being movable between an open position, in which the portions are spaced apart from one another, and a closed position, in which the top and bottom portions are configured to engage a spinal rod disposed there between.

27. An installation device for implanting a spinal connector having top and bottom portions that are adapted to seat a spinal fixation element there between, the installation device comprising:
first and second opposed arms pivotally coupled to one another and movable between an open position, wherein a distal portion of each arm is spaced a distance apart from one another, and a closed position, wherein the distal portion of each arm is adapted to seat and engage a spinal connector disposed there between, the distal portion of each arm being substantially C-shaped to define a connector-receiving recess formed there between when the arms are in the closed position, and the distal portion of each arm including a connector-engaging member formed thereon and adapted to engage a connector positioned there between.

28. The installation device of claim 27, wherein the connector-engaging member on each arm comprises a ridge that is adapted to extend between top and bottom portions of the spinal connector.

29. The installation device of claim 27, further comprising a locking mechanism coupled to each of the first and second arms and adapted to lock the arms in a fixed position relative to one another.

30. The installation device of claim 27, wherein the distal portion of each arm includes an alignment mechanism formed thereon and adapted to align a spinal connector relative to the distal portion of each arm.

31. The installation device of claim 30, wherein the alignment mechanism comprises at least one tab formed on each arm.

32. An installation device for implanting a spinal connector having top and bottom portions that are adapted to seat a spinal fixation element there between, the installation device comprising:
an elongate shaft having a connector-receiving member formed on a distal end thereof and adapted to seat a spinal connector;
a driver shaft rotatably disposed through the elongate shaft and including a mating element formed on a distal end thereof and adapted to mate with a fastening element to rotate the fastening element relative to a spinal connector coupled to the elongate shaft, thereby mating the fastening element to the connector.

33. The installation device of claim 32, wherein the connector-receiving member comprises a housing having a recess formed therein for seating a spinal connector.

34. The installation device of claim 33, wherein the mating element on the driver shaft extends through and distally beyond the housing.

35. The installation device of claim 34, wherein the mating element comprises an elongate shaft, at least a portion of which is asymmetrical for extending into a socket formed in a fastening element.

36. The installation device of claim 35, wherein at least a portion of the elongate shaft that forms the mating element is threaded to mate with corresponding threads formed within a spinal connector.

37. A spinal fixation kit, comprising:
a spinal clamp adapted to seat a spinal fixation element there between, the clamp having at least one bore extending therethrough for receiving a fastening element, the at least one bore including left-handed threads formed in at least a portion thereof; and
a fastening element having a shaft including left-handed threads formed on a least a portion thereof for mating with the left-handed threads formed in at least a portion if the at least one bore.

38. The spinal fixation kit of claim 37, wherein the spinal clamp includes top and bottom portions, and wherein each portion includes a bore formed therein and axially aligned with one another.

39. The spinal fixation kit of claim 37, wherein the fastening element includes a head formed on a proximal end of the shaft.

40. The spinal fixation kit of claim 39, wherein a distal-most end of the shaft of the fastening element includes a socket formed therein for receiving a driver tool.

41. The spinal fixation kit of claim 40, further comprising an installation device having a hollow elongate shaft with a distal, connector-receiving portion adapted to seat the connector, and a driver tool rotatably coupled to the shaft and including a distal end that is adapted to extend through the at least one bore in the connector and into the socket in the fastening element such that rotation of the driver tool is effective to mate the fastening element to the connector.

42. The spinal fixation kit of claim 41, further comprising a spinal anchoring element having at least one thru-bore formed therein, the fastening element being removably matable to the at least one thru-bore in the spinal anchoring element.

43. A spinal fixation system, comprising:
a spinal fixation plate having at least one thru-bore formed therein; and
a spinal connector having top and bottom portions that are connected to one another at a terminal end thereof and that are adapted to retain a spinal rod there between, the spinal connector being configured to engage a superior surface of the spinal fixation plate to mate a spinal rod to the spinal fixation plate.

44. The spinal fixation system of claim 43, further comprising a locking mechanism adapted to lock the top and bottom portions of the spinal connector in a fixed position relative to one another to engage a spinal rod there between, the locking mechanism being further adapted to mate the spinal connector to the spinal fixation plate.

45. The spinal fixation system of claim 44, wherein the fastening element includes left-handed threads formed thereon for mating with corresponding left-handed threads formed within at least a portion of a bore extending through the top and bottom portions of the spinal connector.

46. The spinal fixation system of claim 43, wherein the top and bottom portions are connected to one another by a hinge member formed on a terminal end thereof.

47. The spinal fixation system of claim 43, further comprising a recess formed between the top and bottom portions of the spinal connector for seating a spinal rod.

48. The spinal fixation system of claim 47, wherein the recess has a substantially cylindrical shape.

49. The spinal fixation system of claim 47, wherein the recess extends in a direction substantially parallel to a direction of a terminal end of each of the top and bottom portions of the spinal connector.

50. The spinal fixation system of claim 43, wherein the top and bottom portions are biased to a closed position.

51. The spinal fixation system of claim 43, wherein the locking mechanism extends through the top and bottom portions to lock the top and bottom portions in a fixed position, and the locking mechanism extends at least partially into the at least one thru-bore formed in the spinal fixation plate.

52. The spinal fixation system of claim 51, further comprising a bore formed through each of the top and bottom portions of the spinal connector for receiving the locking mechanism.

53. The spinal fixation system of claim 52, wherein the bore has an elongate slotted configuration.

54. The spinal fixation system of claim 52, wherein the bore is substantially cylindrical.

55. The spinal fixation system of claim 51, wherein the at least one thru-bore in the spinal fixation plate has an elongate slotted configuration.

56. The spinal fixation system of claim 51, wherein the at least one thru-bore in the spinal fixation plate is substantially cylindrical.

57. The spinal fixation system of claim 43, wherein the bottom portion of the spinal connector is fixedly mated to the spinal fixation plate.

58. The spinal fixation system of claim 43, wherein the bottom portion of the spinal connector is integrally formed with the spinal fixation plate.

59. A medical connector device, comprising:
a substantially J-shaped connector body having a first, substantially planar portion with a thru-bore formed therein, and a second, substantially curved portion that extends in a direction substantially transverse to the first portion and that defines a recess for seating a spinal fixation element, the thru-bore in the first portion being positioned to receive a fastening element such that a head of the fastening element is effective to contact a spinal fixation element seated within the recess in the second portion and to lock the spinal fixation element within the recess.

60. A spinal fixation system, comprising:
an elongate spinal fixation element;
a spinal fixation plate having at least one thru-bore formed therein;
a connector member having a planar portion with a bore formed therein and adapted to be aligned with the at least one thru-bore formed in the spinal fixation plate, and a curved portion extending in a direction transverse to the planar portion and defining a recess for seating the elongate spinal fixation element; and
a locking mechanism having a head and a shaft adapted to extend through the bore formed in the planar portion of the connector member, the shaft being adapted to at least partially extend into the at least one thru-bore formed in the spinal fixation plate, and the head being configured to lock the spinal fixation element within the recess in the curved portion of the connector member when the locking mechanism is in a locked position.

61. The spinal fixation system of claim 60, wherein the shaft of the locking mechanism is adapted to mate to the at least one thru-bore in the spinal fixation plate.

62. The spinal fixation system of claim 60, wherein the shaft of the locking mechanism is adapted to extend through the least one thru-bore in the spinal fixation plate and to extend into and engage bone.

63. A spinal fixation system, comprising:
a spinal plate having at least one thru-bore formed therein;
at least one anchoring device having a distal hook portion adapted to engage bone and a proximal receiver head adapted to receive an elongate spinal fixation element, the receiver head being positionable within the at least one thru-bore formed in the spinal plate such that a spinal fixation element disposed within the receiver head can be coupled to the spinal plate, and the spinal plate can be mated to bone.

64. The spinal fixation system of claim 63, wherein the spinal plate is an occipital plate having first and second opposed thru-bores formed therein, and wherein the system includes a first anchoring device having a receiver head disposed within the first thru-bore formed in the occipital plate, and a second anchoring device having a receiver head disposed within the second thru-bore formed in the occipital plate.

65. The spinal fixation system of claim 63, further comprising at least one locking mechanism adapted to be disposed within the receiver head of the at least one anchoring device to lock a spinal fixation element therein.
